(19) United States
(12) Patent Application Publication
(10) Pub. No.: US 2004/0180060 A1
(43) Pub. Date: Sep. 16, 2004

(54) Adjuvanted bovine vaccines

(75) Inventors: Wumin Li, Fort Dodge, IA (US); Hsien-Jue Chu, Fort Dodge, IA (US)

Correspondence Address: WYETH, PATENT LAW GROUP, FIVE GIRALDA FARMS, MADISON, NJ, 07940, US

(73) Assignee: Wyeth, Madison, NJ

(21) Appl. No.: 10/796,925

(22) Filed: Mar. 10, 2004

Related U.S. Application Data
(60) Provisional application No. 60/454,182, filed on Mar. 12, 2003.

(51) Int. Cl. C12P 21/06; A61K 39/00; A61K 39/38

(52) U.S. Cl. 424/184.1

(57) ABSTRACT

The present invention provides a safe and effective vaccine composition against E. coli O157:H7 shedding. An immunogenically active component E. coli O157:H7, an adjuvant such as a metabolizable oil, and a pharmacologically acceptable carrier are formulated into an immunizing vaccine. The invention also provides a method for the prevention or amelioration of E. coli O157:H7 shedding.

This application claims priority from copending provisional Application Serial No. 60/454,182, filed Mar. 12, 2003, the entire disclosure of which is hereby incorporated by reference.

### FIELD OF THE INVENTION

The present invention relates to adjuvanted vaccines for the reduction of E. coli O157:H7 colonization in animals, particularly cattle, methods for their preparation, and methods of administering same to animals, particularly cattle, so as to prevent shedding thereof.

### BACKGROUND OF THE INVENTION

E. coli O157:H7 is a virulent and common food borne pathogen, and thus E. coli O157:H7 infections are a source of serious concern to human health. Human illness associated with infection by E. coli O157:H7 has been reported with increasing frequency since 1982. The epidemiological link between human disease and consumption of bovine products has been supported by the isolation of E. coli O157:H7 from calf or adult bovine feces collected from farms or feedlots in the United States, Canada and other countries. The ingestion of contaminated beef or other meat products, and not person to person spread, is the chief source of human infection.

E. coli O157:H7 colonizes the intestines of ruminants and other mammals and generally does not cause overt disease in these animals. The shedding of the E. coli O157:H7 into feces of colonized animals serves as a source of E. coli O157 infection in humans. It is important, therefore, to eradicate or reduce E. coli O157:H7 colonization and shedding in animals, particularly cattle, to prevent human infection. Oral inoculation of calves with E. coli O157:H7 has been demonstrated to induce prompt and sustained increase in serum antibodies to LPS and neutralizing antibodies to verotoxins. Attempts have also been made to reduce E. coli shedding from cattle by a brief period of feed-changing from grain to hay. This feed-changing method, however, is unable to totally eliminate environmental feces contamination, because it is unlikely that American cattle will ever be fed diets consisting only of hay.

Because of the bulk processing of slaughtered cattle and the low number of E coli O157:H7 (10-100) necessary to infect a human, E. coli O157:H7 remains a serious health problem. Research has focused on improved methods for detecting and subsequently killing E. coli O157:H7 at slaughter, altering the diet of cattle to reduce the number of intestinal E. coli O157LH7, and immunizing animals to prevent E. coli O157:H7 shedding. Still though, occasionally, and with sometimes disastrous economic and public health consequences, E. coli O157:H7 slips through the net, and, in combination, almost always, with human error (improper cooking or cross-contamination), wreaks havoc. For the last several years, scientists, cattle producers, journalists, association personnel, government representatives and packing plant officials have indicated that there is a mandate for farmers/ranchers to assume responsibility for actively attempting to prevent (or at least minimize) the risk that slaughtered cattle would carry food borne pathogens into the packing plant, on or in their bodies. It has been postulated that: (a) cleaner animals would reduce the odds of pathogen presence on carcasses, cuts and final beef products, (b) Good Management Practices, or Good Production Practices, would be helpful for presenting cleaner animals for slaughter, and (c) the impact of selected interventions and management practices in minimizing presence of food borne pathogens on and in slaughtered cattle should be investigated.

The Texas Cattle Feeders Association have reported that a product called Tasco™, made from a brown seaweed found in the North Atlantic Ocean, reduced E. coli O157:H7 in cattle by 300% when included in the ration for 14 days prior to slaughter. CALF News (2002) reported that a new feed ingredient that contains probiotics or so-called “good bacteria” (in fact, strains of Lactobacillus acidophilus) can reduce the presence of E. coli O157:H7 in live cattle by as much as 50% based on studies funded by American Meat Institute Foundation. Zhao et al. (1998) reported results indicating that selected probiotic bacteria (including non-Enterohemorrhagic Escherichia coli and Proteus mirabilis) administered to cattle prior to exposure to E. coli O157:H7 can reduce the level of carriage of E. coli O157:H7 in most animals; L-Pharma, Inc. has now commercialized a probiotic for cattle based on that study.

Nonetheless, it remains a challenge to produce a vaccine to effectively prevent E. coli O157:H7 colonizations in ruminant animals, particularly bovines, that can be passed through their carcasses into the human food supply.

### SUMMARY OF THE INVENTION

The present invention provides a vaccine composition comprising an immunogenically active component selected from the group consisting of inactivated or killed whole or subunit E. coli O157:H7 antigens, in combination with a metabolizable oil and aluminum hydroxide adjuvant.

The metabolizable oil is utilized in the vaccine composition is an immunogenically stimulating amount, along with other conventional vaccine excipients.

In a further embodiment of the invention, the vaccine composition comprises at least 1×10⁹ cells per unit dose of inactivated E. coli O157:H7, or a component thereof, and about 5% to 10% vol/vol of an adjuvant comprising about 3-8%, preferably 5%, of a metabolizable oil and about 10-25%, preferably 15%, aluminum hydroxide.

A particularly preferred embodiment of the invention is a vaccine composition for calves, comprising at least two dosage units of killed or inactivated E. coli O157:H7, wherein each said dosage unit comprises about at least 1×10⁹ of said bacterin and about 5 to 25% vol/vol of an adjuvant, said adjuvant comprising at least one metabolizable oil, and aluminum hydroxide, and further wherein said dosage unit comprises a pharmacologically acceptable carrier.

Further, objects and features of the invention will become apparent from the detailed description and the claims set forth herein below.

### DETAILED DESCRIPTION OF THE INVENTION

In general, the problem with designing a new vaccine is that a live bacterial vaccine may potentially lack sufficient safety in a given target host, and that a killed or inactivated bacterial vaccine may potentially lack the ability to stimulate a sufficiently effective immunologic response. Commonly, an adjuvant or immunogenically stimulating compound is used in combination with a killed or inactivated bacteria in a vaccine composition to obtain acceptable efficacy. However, safety to the target host is often compromised by the addition of an adjuvant. For example, pregnant animals many times have been known to have a significantly higher rate of miscarriage after being administered a killed or inactivated bacteria vaccine that contains an adjuvant. Additionally, in food animals, it is highly desirable to minimize injection site reactions which adversely impact the meat quality of an animal which is sold for food consumption.

It has now been found that when a suitable adjuvant, e.g., a metabolizable oil, is used in combination with an immunogenically active component as described herein, the resultant E. coli O157:H7 vaccine composition is safened for use, and is particularly useful in bovines. Thus, the invention achieves the concomitant goals of effective immunization and safety, with minimal injection site reactions that would be deleterious to meat quality.

A safe and effective vaccine composition comprises: an immunogenically active component selected from the group consisting of an inactivated or killed whole, or subunit of, E. coli O157:H7, together with a suitable adjuvant. Such a vaccine will effectively prevent colonization of a ruminant animal, thereby reducing or eliminating its potential to shed the E. coli O157:H7 into the human food supply.

As used herein the term “immunogenically active” means the ability to stimulate an immune response, i.e., to stimulate the production of antibodies, particularly humoral antibodies, or to stimulate a cell-mediated response. The amount of the immunogenically active component which is effective and immunizing may vary and is any amount sufficient to evoke an immune response and provide immunological protection against E. coli O157:H7 colonization. The amount of immunogenically active component per dosage unit is preferably at least about 1×10⁹ cells. These amounts are suitable for inactivated or killed whole cell, or subunit of, antigen.

The immunogenically active component can be whole or subunit E. coli O157:H7 that has been isolated from colonized animals using conventional techniques. It may also be derived from any of a number of available isolates of E. coli O157:H7, such as those obtainable from various national and international culture collections which maintain a depository for organisms such as E. coli O157H7. At the American Type Culture Collection (ATCC), for example, the E. coli O157:H7 has been deposited, inter alia, under ATCC Nos. 35150, 43888, 43889, 43890, 43894, and 43895. At the Centro Venezolano de Colecciones de Microorganismos, Instituto de Biologia Experimental, Universidad Central de Venezuela the E. Coli O157H7 has been deposited under CVCM815. At Collection de L'lnstitut Pasteur, Institut Pasteur, the E. coli O157:H7 has been deposited under CIP759. At the Bioresource Collection and Research Center, Food Industry Research and Development Institute, E. coli O157:H7 has been deposited under BCRC59. Also, PCT WO 00/04922 describes particular subunit E. coli O157:H7 antigens prepared from O-specific polysaccharide of E coli O157:H7.

At least one dosage unit per animal is contemplated herein as a vaccination regimen. Two or more dosage units may be especially useful. A dosage unit may typically be about 1 to 2 milliliters, with each dosage unit containing the heretofore described quantity of bacteria or bacterial component. The skilled man will recognize that a particular quantity of vaccine composition per dosage unit, as well as the total number of dosage units per vaccination regimen, may be optimized, so long as an effective immunizing amount of the bacterin or a component thereof is delivered to the animal.

The E. coli O157:H7 vaccine composition of the present invention contains a suitable adjuvant which most preferably contains a metabolizable oil as one of its components. As used herein the term “adjuvant” refers to any component which improves the body's response to a vaccine or an immunogen. The adjuvant will typically comprise about 0.1 to 50% vol/vol of the vaccine formulation of the invention, preferably about 1 to 50% of the vaccine, more preferably about 1 to 20%, particularly 1 to 10% vol/vol thereof. Amounts of about 5 to 15% vol/vol³ are even more preferred.

The adjuvant utilized in the vaccine composition includes at least one immunostimulating oils which is metabolizable by the target species. Metabolizable oils suitable for use in the composition of the invention include oil emulsions, e.g., SP oil (hereinafter described), Emulsigen (MPV Laboratories, Ralston, NZ), Montanide 264,266,26 (Seppic SA, Paris, France), as well as peanut oil and other vegetable-based oils, squalane (shark liver oil) or other metabolizable oils which are suitable for use an adjuvant in veterinary vaccine practice.

The adjuvant composition preferably comprises, in addition to the metabolizable oil, one or more wetting or dispersing agents in amounts of about 0.1 to 25%, more preferably about 1 to 10%, and even more preferably about 1 to 3%, by volume of the adjuvant. Particularly preferred as wetting or dispersing agents are non-ionic surfactants. Other components of the adjuvant may include such preservative compounds as benzyl alcohol formalin and thimerosal in amounts of up to about 1% vol/vol of the adjuvant.

A particularly preferred adjuvant is a metabolizable oil formulation referred to as SP oil. As used in the description and examples, the term “SP oil” designates an oil emulsion comprising a polyoxyethylene-polyoxypropylene block copolymer, squalane, polyoxyethylene sorbitan monooleate and a buffered salt solution. In general, the SP oil emulsion will comprise about 1 to 3% vol/vol of block copolymer, about 2 to 6% vol/vol of squalane, more particularly about 3 to 6% of squalane, and about 0.1 to 0.5% vol/vol of polyoxyethylene sorbitan monooleate, with the remainder being a buffered salt solution.

In a highly preferred vaccine composition of the present invention, the metabolizable oil is utilized in conjunction with aluminum hydroxide gel, preferably in an amount of about 10-20% vol/vol, and most preferably in an amount of about 15% vol/vol. This combination of SP oil and aluminum hydroxide provides an especially useful vaccine in that both systemic and local immune effects are induced in the vaccinated ruminant. Another surprising feature is that this combination of adjuvants has shown, in some cases, significant safety improvement with certain antigen forms.

When utilized, immunogenically stimulating amounts of SP oil as adjuvant in the vaccine composition of the invention may vary according to the immunogenically active component, the degree of potential infectious exposure, method of administration of the vaccine composition, the age and size of the bovine, or the like. In general, amounts of about 1% to 50% vol/vol of the vaccine composition are suitable, preferably about 4% to 10% vol/vol, and more preferably about 4% to 5% vol/vol of SP oil.

Pharmaceutical (or pharmacologically) acceptable carriers suitable for use in the vaccine composition of the invention may be any conventional liquid carrier suitable for veterinary pharmaceutical compositions, preferably a balanced salt solution or other water-based solution suitable for use in tissue culture media. Other available carriers may also be utilized.

Additional excipients available in the art may also be included in the vaccine composition according to the various embodiments heretofore described. For example, pH modifiers may be utilized.

The components of the vaccine composition of the invention as heretofore described, including the carrier, may be combined together using available techniques.

In addition to the immunogenically active component of E. coli O157:H7 as described hereinabove as active ingredient, it is contemplated that the vaccine composition of the invention may also contain other active components such as an antipathogenic component directed against Salmonella dublin or Salmonella typhimurium or the like or a combination thereof. The quantities of one or more of these bacteria may be determined from efficacy literature, or determined using available techniques.

In one embodiment of the invention the immunogenically active component of the invention may be conjugated to suitable biological compounds such as polysaccharides, peptides, proteins, or the like, or a combination thereof.

In a preferred embodiment of the invention, the inventive vaccine composition may be formulated in dosage unit form as heretofore described to facilitate administration and ensure uniformity of dosage. Formulation may be effected using available techniques, such as those applicable to preparations of emulsions.

The inventive vaccine composition may be administered parenterally, for example, intramuscularly, subcutaneously, intraperitoneally, intraderrrially or the like, preferably subcutaneously.

In actual practice, the vaccine composition of the invention is administered parenterally, subcutaneously or by other available means, preferably parenterally, more preferably subcutaneously, in effective amounts according to a schedule which may be determined by the time of anticipated potential exposure to a carrier of the E. coli O157:H7. In this way, the treated animal may have time to build immunity prior to the natural exposure. By way of non-limiting example, a typical treatment schedule or dosing regimen may include parenteral administration, preferably subcutaneously injection of one dosage unit, at least about 2-8 weeks prior to potential exposure. At least two administrations are preferred, for example one dosage unit at about 8 weeks prior to potential exposure to the bacterin and a second dosage unit at about 3 -5 weeks prior to potential exposure of the treated animal. As heretofore described, a dosage unit will typically be within the range of about 0.1 to 10 milliliters of vaccine composition containing the amounts of active and percentages of adjuvant and inactive(s) as previously described. A dosage unit within the range of about 0.5 to 5 milliliters is perhaps more preferred, with about 1 to 2 milliliter(s) being particularly preferred.

For a clearer understanding of the invention, the following examples are set forth below. These examples are merely illustrative and are not understood to limit the scope or underlying principles of the invention in any way. Indeed, various modifications of the invention, in addition to those shown and described herein, will become apparent to those skilled in the art from the following examples and the foregoing description. Such modifications are also intended to fall within the scope of the appended claims.

### EXAMPLES

### Example 1

### Preparation of Vaccine

FORMULATION OF SP OIL

| INGREDIENT DESCRIPTION | Volume |
|---|---|
| Polyoxyethylene-polyoxypropylene block copolymer | 20.0 ml |
| (Pluronic ® L121, BASF, Mt. Olive, NJ) | |
| Squalane (Kodak, Rochester, NY) | 40.0 ml |

The ingredients are mixed and homogenized until a stable mass or emulsion is formed. Prior to homogenization, the ingredients or mixture may be autoclaved. The emulsion may be further sterilized by filtration

### Vaccine Formulation Bovine E. coli O157:H7 Bacterin

DOSE VOLUME: 2 ML/DOSE

| | | | | Volume | | |
|---|---|---|---|---|---|---|
| | Stock | | | Stock/mL | Total Vol./15 | |
| Component | Concentration | Amount/mL | Amount/Dose | Vaccine | 15,000 mL | |
| E. coli O157: H7 | 3.86 × 10⁹ | 5 × 10⁸ cells | 1 × 10⁹ cells | 0.129 | 1,943 | mL |
| ATCC 43889 | cells/mL (1×) | | | | | |
| ALOH (Sterile gel) | N/A | 15% v/v | 15% v/v | 0.15 | 2,250 | mL |
| *SP Oil (with | N/A | 5% v/v | 5% v/v | 0.05 | 750 | mL |
| Thimerosal) | | | | | | |
| 5% Thimerosal | N/A | 1:2500 | 1:2500 | N/A | 5.25 | mL |
| 0.01 M PBS | N/A | N/A | N/A | N/A | 10,051.75 | mL |

| | | |
|---|---|---|
| Total | 15,000 | mL |

13.8 liters were harvested from fermentation with concentration at 3.86 × 10\hat{ }9 cells/mL
13.8 liters were harvested from fermentation with concentration at 3.86 × 10\hat{ }9 cells/mL
*750 mL of SP oil contains 0.75 mL of 5% Thimerasol (750 mL × 0.001 = 0.75 mL)
6 mL − 0.75 mL = 5.25 mL additional amount need

Blending Sequence:

1. Mix the inactivated bacteria for at least 30 minutes @150-200 rpm to ensure mixed well

2. Take 3,000 mL of the mixed bacteria and centrifuge at 10,000 rpm for 30 minutes (keep the rest antigen stock at 4 C)

3. Collect pellet and resuspend the pellet with 0.01M PBS, QS to 3,000 mL and mix well

4. Take 971.5 mL of the resuspended cells and add 2,028.5 mL of 0.01M PBS to make the total volume at 3,000 mL. This is fraction A.

5. Take another 971.5 mL of the resuspended cells and add 2,028.5 mL of 0.01M PBS to make the total volume at 3,000 mL. This is fraction B.

6. Add 2,250 mL of ALOH gel into fraction A and mix this combination for 1 hour at 150-200 rpm

7. Add 750 mL of SP oil into fraction B and mix this combination for 1 hour at 150-200 rpm

8. Combine the above fraction A and fraction B and mix the combination for 1 hour @150-200 rpm

9. Add 5.25 mL of 5% Thimerosal and Q.S the volume to 14,800 mL with 0.01M PBS

10. Mix the vaccine at least 30 minutes @150-200 rpm

11. Check the pH and adjust pH to 7(+0.2) if needed

12. After the pH is adjusted, QS the vaccine to 15,000 mL with 0.01M PBS and mix it for at least another 30 minutes.

13. Fill and label the vaccine

### Example 2

### Evaluation of Serological Response in Cattle Following Vaccination with Adjuvanted or Unadjuvanted E. coli O157:H7 Vaccines and the Safety of the Test Vaccines in Cattle

Twenty-four healthy mixed breed cattle obtained from commercial sources are used in the study. Their age range is 6-12 months at first vaccination, and both male and female animals are used. The cattle are group housed in housing meeting applicable animal welfare regulations. Water and food is available ad lib. All animals are treated as deemed necessary by the plant veterinarian after consultation with the study director. Treatment(s) before and during the study are documented. Animals requiring antibiotics or potentially immunosuppressive drugs are removed from the study.

Vaccine compositions are formulated and tested for sterility and laboratory animal safety as specified in 9 CFR §§ 113.26 and 113.33. Vaccines are stored at 2-7° C. Calves are randomly divided into groups of six animals each. Group 6 is vaccinated with a conventionally adjuvanted vaccine. Group 7 is vaccinated with a vaccine adjuvanted in accordance with the present invention and Group 5 is held as unvaccinated controls. Calves are vaccinated with a 2 mL dose with the appropriate vaccine by the subcutaneous route. A second dose is administered in 3-4 weeks, and a third dose is administered after a further 3-4 weeks. Calves are bled at the time or the first and second dose and weekly thereafter until four weeks post third vaccination. Each serum sample is evaluated for antibody response.

Serum analysis is analyzed by statistical methods to determine differences in antibody response. ELISA Titers are determined to assess vaccine response, and results are averaged.

Injection sites are observed for three days following each vaccination. If any injection site reactions are seen, the cattle are then observed up to 14 days post vaccination or until the reaction has dissipated. Injection site reactions are measured in three dimensions (length, width and height). A daily reaction score is calculated by L×W×H. Total reaction scores are analyzed by Mann Whitney Rank Sum. The level of significance is set at p<0.05.

Results are as follows:
Serology: ELISA TITERS
Control: Group 5
Standard Adjuvant: Group 6
Invention SP Oil Oil/Aluminum Hydroxide Adjuvant: Group 7

| | | 0 days post first | 14 days post third |
|---|---|---|---|
| Vaccine group | Calf# | vaccination | vaccination |
| 5 | 283 | 640 | 1280 |
| 5 | 291 | 640 | 640 |
| 5 | 367 | 640 | 640 |
| 5 | 368 | 640 | 640 |
| 5 | 369 | 640 | 640 |
| | | 640 | 735 |
| 6 | 389 | 640 | 640 |
| 6 | 277 | 640 | 640 |
| 6 | 292 | 2560 | 2560 |
| 6 | 379 | 320 | 640 |
| | | 735 | 868 |
| 7 | 390 | 640 | 1280 |
| 7 | 384 | 1280 | 2560 |
| 7 | 294 | 320 | 1280 |
| | | 573 | 1184 |

Results: The animals of Group 7 show enhanced immunogenic response over those of the control group and Group 6 based on the levels of the ELISA titers fourteen days post third vaccination.

Reaction Scores which assess Injection site reactions:

| | −1dpv2 | 0dpv2 | 1dpv2 | 2dpv2 | 3dpv2 | 4dpv2 | 5dpv2 | 6dpv2 | 7dpv2 | 10dpv2 | 11dpv2 |
|---|---|---|---|---|---|---|---|---|---|---|---|
| CONTROL | 0 | 0 | 0.0 | 0.0 | 0.0 | 0.0 | 0.0 | 0.0 | 0.0 | 0.0 | 0.0 |
| Conventional | 0 | 0 | 68.4 | 58.0 | 31.9 | 30.8 | 19.0 | 9.8 | 10.1 | 6.6 | 1.5 |
| Invention Adjuvant | 0 | 0 | 25.4 | 61.5 | 43.3 | 52.1 | 61.3 | 24.9 | 15.3 | 1.2 | 2.8 |

−1dpv2 = assessment of injection site on day before second vaccination
0dpv2 = assessment of injection site on day of second vaccination
1dpv2 = assessment of injection site one day post second vaccination
2dpv2 = assessment of injection site two days post second vaccination
3dpv2 = assessment of injection site three days post second vaccination
4dpv2 = assessment of injection site four days post second vaccination
5dpv2 = assessment of injection site five days post second vaccination
6dpv2 = assessment of injection site six days post second vaccination
7dpv2 = assessment of injection site seven days post second vaccination
10dpv2 = assessment of injection site ten days post second vaccination
11dpv2 = assessment of injection site eleven days post second vaccination

adjuvanted in accordance with the present invention, with significantly higher immunogenic responses.

### Example 3

### Field Study

The vaccine composition of Example 1 was utilized in a commercial feedlot in a two-month study to assess and compare the effectiveness of various interventions to reduce the prevalence of E. coli O157 in feedlot cattle. The E. coli of Example 1 was administered twice during the Study at a one-month interval. Thirty days following the last vaccination USDA-FSIS granted slaughter permits for the vaccinated cattle. The vaccine stimulates the host immune system, specifically for both T cells and B cells to elicit humoral antibody and some CMI factors.

Hide and fecal samples were collected from 25 cattle per pen within 48 h of transport to a slaughter facility. Following collection, samples were transported to the laboratory for analysis. Data from the E. coli 0157 analyses were reported as percentages of hide, fecal and hide or fecal samples testing positive for the pathogen, divided by total samples collected per pen. Since both the hide and fecal samples came from the same animal, the researchers analyzed the data such that, if either the hide or the fecal sample was positive, the animal was considered positive. Differences in percentage positive samples among treatments were determined using a chi-square goodness of fit test (SAS Inc., Cary, N.C.). The vaccine was found to reduce pathogen prevalence by 20.3% on hide samples, and by 31.1% in fecal samples. When combined with other intervention strategies, such as treatment with Lactobacillus acidophilis or a neomycin medicated feed supplement, the vaccine provides additional reduction in antigen shedding.

What is claimed is:

1. A vaccine composition which comprises: an immunogenically active component selected from the group consisting of inactivated or killed whole or subunit E. coli O157:H7, or mixtures thereof; a metabolizable oil adjuvant; and optionally a pharmaceutically acceptable carrier.

2. The composition according to claim 1 wherein the immunogenically active component is an inactivated whole or subunit E. coli O157:H7.

3. The composition according to claim 2 wherein the immunogenically active component is an inactivated whole E. coli O157:H7.

4. The composition according to claim 2 wherein the immunogenically active component is subunit E. coli O157:H7.

5. The composition according to claim 3 wherein the adjuvant comprises 0.1 to 50% vol/vol of the vaccine composition.

6. The composition according to claim 4 wherein the adjuvant comprises 0.1 to 50% vol/vol of the vaccine composition.

7. The composition according to claim 5 wherein the adjuvant comprises a metabolizable oil and aluminum hydroxide gel.

8. The composition according to claim 6 wherein the adjuvant comprises a metabolizable oil and aluminum hydroxide gel.

9. The composition according to claim 5 wherein the adjuvant comprises from 1 to 50% vol/vol of metabolizable oil.

10. The composition according to claim 6 wherein the adjuvant comprises from 1 to 50% vol/vol of metabolizable oil.

11. The composition according to claim 5 wherein the metabolizable oil is squalane.

12. The composition according to claim 6 wherein the metabolizable oil is squalane.

13. The composition according to claim 5 wherein the adjuvant further comprises one or more wetting agents and/or dispersing agents in an amount of from about 0.1 to 25% vol/vol of the adjuvant.

14. The composition according to claim 6 wherein the adjuvant further comprises one or more wetting agents and/or dispersing agents in an amount of from about 0.1 to 25% vol/vol of the adjuvant.

15. The composition of claim 13, wherein said wetting or dispersing agents are selected from the group consisting of non-ionic surfactants.

16. The composition of claim 14, wherein said wetting or dispersing agents are selected from the group consisting of non-ionic surfactants.

17. The composition of claim 17, wherein said non-ionic surfactants are selected from the group consisting of polyoxyethylene/polyoxypropylene block copolymers and polyoxyethylene esters.

18. The composition of claim 18, wherein said non-ionic surfactants are selected from the group consisting of polyoxyethylene/polyoxypropylene block copolymers and polyoxyethylene esters.

19. The composition according to claim 17 wherein said immunogenically active component is present in sufficient quantity to provide at least 1×10⁹ cells per unit dose.

20. A method for reducing shedding of E. coli O157 in an animal which comprises treatment of the animal with a composition according to claim 1.

21. A method according to claim 20 which further comprises treatment of the animal with a Lactobacillus acidophilis or neomycion medicated feed supplement.
